(19) United States
(12) Patent Application Publication
(10) Pub. No.: US 2018/0317835 A1
(43) Pub. Date: Nov. 8, 2018

(54) SYSTEM AND METHOD FOR DETECTING CONTRACTIONS

(71) Applicant: Hera Med Ltd., Netanya (IL)

(72) Inventors: David GROBERMAN, Even-Yehuda (IL); Tal SLONIM, Rishon Lezion (IL); Joel ROTEM, Kfar-Sirkin (IL); Michael NENNER, Tel-Aviv (IL)

(73) Assignee: Hera Med Ltd., Netanya (IL)

(21) Appl. No.: 15/756,064

(22) PCT Filed: Aug. 29, 2016
(86) PCT No.: PCT/IL2016/050946

§ 371 Date: Feb. 28, 2018

Related U.S. Application Data
(60) Provisional application No. 62/212,616, filed on Sep. 1, 2015.

(51) Int. Cl. A61B 5/00; A61B 5/0488

(52) U.S. Cl. CPC A61B 5/4356; A61B 5/053; A61B 5/04882; A61B 5/4362

(57) ABSTRACT

A method of detecting contractions in a user, the method may include providing alternating current with a determined frequency, through a first path in an abdominal tissue, between at least a first pair of electrodes positioned apart from each other over a first abdominal surface of the user. The method may further include repeatedly measuring a response of the abdominal tissue to excitation by the alternating current passing between at least a pair of electrodes, identifying at least one muscle contraction according to the changes in the response, and determining occurrence of uterine contractions based on at least one characteristic of the at least one muscle contraction. A system for detecting contractions in a user may include an alternating current generator, at least two electrodes, and a processor, operatively coupled to the at least two electrodes.

### FIELD OF THE INVENTION

The present invention relates to monitoring of women during labor. More particularly, the present invention relates to systems and methods for processing bio-impedance and other signals to detect uterine contractions.

### BACKGROUND OF THE INVENTION

Premature birth is the leading cause (about 85%) of infant mortality, and improved uterine contraction monitoring technology holds the potential to advance prenatal care, in order to provide obstetricians with a way to diagnose if a user is at risk of preterm labor or other problems during pregnancy As different women have different sensations of contraction (and some women don't feel them at all), as well as some women begin to feel contraction from the sixth week of the pregnancy, such monitoring is critical. However, relying on the feeling of the user is not sufficient for early detection of uterine contraction. Currently monitoring of uterine contractions is usually carried out with a tocodynamometer (or pressure spring) over the upper abdomen of a woman prior to and/or during labor, to measure the abdominal pressures. The tocodynamometer needs to be precisely positioned for monitoring since every movement of the user may cause a contraction of the muscles of the abdomen and be mistaken for a uterine contraction.

A contraction of the uterine wall begins at the top of the uterus and moves downward, towards the woman's pelvis. The tocodynamometer is typically placed over the fundus and secured with an elastic belt. Typically contractions are monitored at the hospital, together with monitoring of the heart rate of the fetus using a separate sensor. It should be noted that in contrast to the heart rate of the fetus, uterine contractions are not constantly present, brief and non-rhythmic and therefore, are typically harder to monitor, for example, a single contraction may last up to a minute, and contractions may have a frequency of once an hour.

However, the tocodynamometer can be uncomfortable for some users (e.g. women in labor or pre-labor) to wear. Additionally, abdominal pressure changes can be harder to detect at early stages of the pregnancy and on women suffering for overweight (e.g. obese). Furthermore, it is necessary to sterilize the belt and sensors so that domestic use cannot be easily achieved. Another disadvantage of the tocodynamometer is that user's movement may change the position of the belt and sensors and may affect the readings of the device. In general, the tocodynamometer is inaccurate. The alternative invasive intrauterine pressure catheter (IUPC) is more reliable and adds contraction pressure information, but requires ruptured membranes and introduces infection and abruption risks. Thus, IUPC cannot be used by untrained caretakers. Another method for contraction monitoring is Electrohysterography (EHG) which monitors the electrical activity of the uterus through electrodes placed on the maternal abdomen. However, currently used methods are less compatible for home use and are passive and thus are expected to be less efficient in early stages of the pregnancy. Furthermore existing methods and systems are not adaptable to the user.

The above solutions require placement by a trained professional at a particular position. Therefore, there is a longstanding need for a uterine contraction monitoring solution to provide reliable, easy to use and continuous monitoring.

### SUMMARY OF THE INVENTION

There is thus provided, in accordance with some embodiments of the invention, a method of detecting contractions in a user, the method may comprise providing alternating current with a determined frequency, through a first path in an abdominal tissue, between at least a first pair of electrodes positioned apart from each other over a first abdominal surface of the user, repeatedly measuring a response of the abdominal tissue to excitation by the alternating current passing between the at least first pair of electrodes, identifying at least one muscle contraction according to the changes in the response, and determining occurrence of uterine contractions based on at least one characteristic of the at least one muscle contraction. In some embodiments, the response is measured in at least one of bio-impedance value, reactance value and phase.

In some embodiments, the method further comprises repeatedly measuring and recording a voltage drop between at least two electrodes of the at least first pair of electrodes, through time. In some embodiments, the first path is perpendicular to a longitudinal axis of a user wherein the longitudinal axis is directed from the top of the head of the user towards the pelvis of the user.

In some embodiments, the method further comprises determining at least a first muscle contraction value, the first contraction value indicative of a magnitude of contraction of the muscle in a first direction, determining at least a second muscle contraction value, the second contraction value indicative of the magnitude of contraction of the muscle in a second direction, and determining occurrence of uterine contraction when the first value is bigger than a first contraction threshold value, and the at least second contraction values is bigger than a second contraction threshold value.

In some embodiments, the method further comprises determining at least a first muscle contraction value, the first contraction value indicative of a magnitude of contraction of the muscle in a first direction, determining at least a second muscle contraction value, the second contraction value indicative of the magnitude of contraction of the muscle in a second direction, comparing at least the first muscle contraction value and the second muscle contraction value, and determining occurrence of uterine contraction when the difference between the first contraction value and the second contraction value is smaller than a predefined value.

In some embodiments, determining the frequency comprises providing at least a first alternating current with a first frequency, between the two electrodes of the at least first pair of electrodes, providing at least a second alternating current with a second frequency, between two electrodes of the at least first pair of electrodes, for each of the at least first and second frequencies, calculating phase difference between the alternating current provided at a first electrode of the at least first pair of electrodes and the alternating current received at a second electrode of the at least first pair of electrodes, and selecting a frequency from the at least first and second frequency, for which the calculated phase difference is maximal, as the determined frequency.

In some embodiments, the determined frequency is in the range of 10 kHz-100 kHz. In some embodiments, the determining of the frequency is repeated every predefined time interval. In some embodiments, the method further comprises determining uterine contraction intensity and duration.

In some embodiments, the response is measured in bio-impedance values, and wherein occurrence of a muscle contraction is determined when the change in bio-impedance between a first bio-impedance value and the at least one second bio-impedance value indicates a decrease in bio-impedance between the electrodes of the at least first pair of electrodes.

In some embodiments, the method further comprises presenting an indication on an output device, to indicate at least one of: an occurrence of a uterine contraction, an intensity of the uterine contraction, and a duration of the uterine contraction.

In some embodiments, the method further comprises monitoring movement of the user, with at least one motion sensor, calculating a movement intensity value, wherein the calculation of the movement intensity value is based on at least one of: acceleration of the at least one motion sensor and change in inclination of the at least one motion sensor, and wherein the determination of occurrence of uterine contraction further requires that the calculated movement intensity value is within a predefined range.

In some embodiments, the determination of occurrence of uterine contraction further requires providing the alternating current with the determined frequency, over a second path between at least a second pair of electrodes positioned apart from each other over a second abdominal surface of the user, wherein the second abdominal surface is closer to the pelvis of the user than the first abdominal surface, calculating a change in tissue response over the second path along time, and determining occurrence of uterine contractions when the change in response over the first path is indicative of uterine contraction, the change in response over the second path is indicative of uterine contraction, and the change in response over the first path occurs at a predefined time interval prior to the change in response over the second path.

There is also provided, in accordance with some embodiments of the invention, a contraction detection system, comprising an alternating current generator configured to provide an alternating current in a determined frequency, through a first path in an abdominal tissue, between at least two electrodes configured to be positioned apart from each other over an abdominal surface of a user, and a processor, operatively coupled to the at least two electrodes, the processor configured to: repeatedly measure and record on a memory, a response of the abdominal tissue to excitation by the alternating current passing between the at least two electrodes, identify at least one muscle contraction according to the changes in the response, and determine occurrence of uterine contractions based on at least one characteristic of the at least one muscle contraction.

In some embodiments, the system further comprises at least one motion sensor, operatively coupled to the at least two electrodes and configured to allow detection of movement of the user. In some embodiments, the at least one motion sensor is selected from a group consisting of accelerometer, gyroscope and microelectromechanical sensor.

In some embodiments, the system further comprises an output device, in active communication with the processor, the output device configured to present at least one of: uterine contraction occurrence, uterine contraction intensity, and uterine contraction duration.

In some embodiments, the output device is a mobile device, connected to the processor via a wireless communication channel. In some embodiments, the at least two electrodes are removably attachable to an abdominal surface of a user. In some embodiments, the at least two electrodes are embedded in a disposable patch. In some embodiments, the determined frequency is in the range of 10 kHz-100 kHz.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a schematic illustration of a uterine contraction detection system, according to some embodiments of the invention;

FIG. 1B schematically illustrates an enlarged view of a monitoring unit of the uterine contraction detection system, according to some embodiments of the invention;

FIG. 2 schematically illustrates a disposable patch, according to some embodiments of the present invention;

FIG. 3 shows examples of phase as function of frequency measurement results that assist in determination of optimal operation frequency, according to some embodiments of the present invention;

FIGS. 4A and 4B are flowcharts of a method of detecting uterine contractions, according to some embodiments of the present invention; and

FIG. 5 is a flowchart of a method of frequency calibration, according to some embodiments of the present invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of embodiments of the invention. However it will be understood by those of ordinary skill in the art that the embodiments of the invention may be practiced without these specific details. In other instances, well-known methods, procedures, and components have not been described in detail so as not to obscure the embodiments of the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

It should be noted that every tissue has a different electrical impedance, and conductivity of muscle tissue is substantially greater than that of skin or fat (covering the uterus), and therefore a current passing through the patient's abdomen may travel through the uterine wall and be affected by changes therein, through a path of minimal resistance. Since the uterus is a large smooth muscle with high liquid content (e.g. over 75 percent), an electrical current passing through the abdomen may pass the uterine wall, especially during later stages (e.g. the second half) of pregnancy when the uterine wall becomes larger and thicker (although stretched due to the growth of the fetus, and uterus) while the rectus abdominis muscles are stretched and become thinner. Therefore, a current passing through the exterior surface of the abdomen may be utilized for monitoring changes in bio-impedance of the tissue and other changes in signal behavior (e.g. changes in phase) in order to detect uterine contractions.

It should be noted that the following layers cover the uterus: a thick uterine wall (e.g. being mostly muscles), additional tissue layer (e.g. fascia transversalis or peritoneum), a thin muscle layer (e.g. being mostly rectus abdominis), a fatty layer (e.g. camper's fascia or scarpa's fascia) and a thin skin layer (e.g. skin and subcutaneous tissue). During later stages of pregnancy, the abdominal muscles are usually stretched and are therefore relatively thin at the monitored area, wherein the uterus may grow thicker (though stretched as well) as much to tear some muscle tissue, and thus such abdominal muscles may have little to no affect to monitoring of uterine contractions.

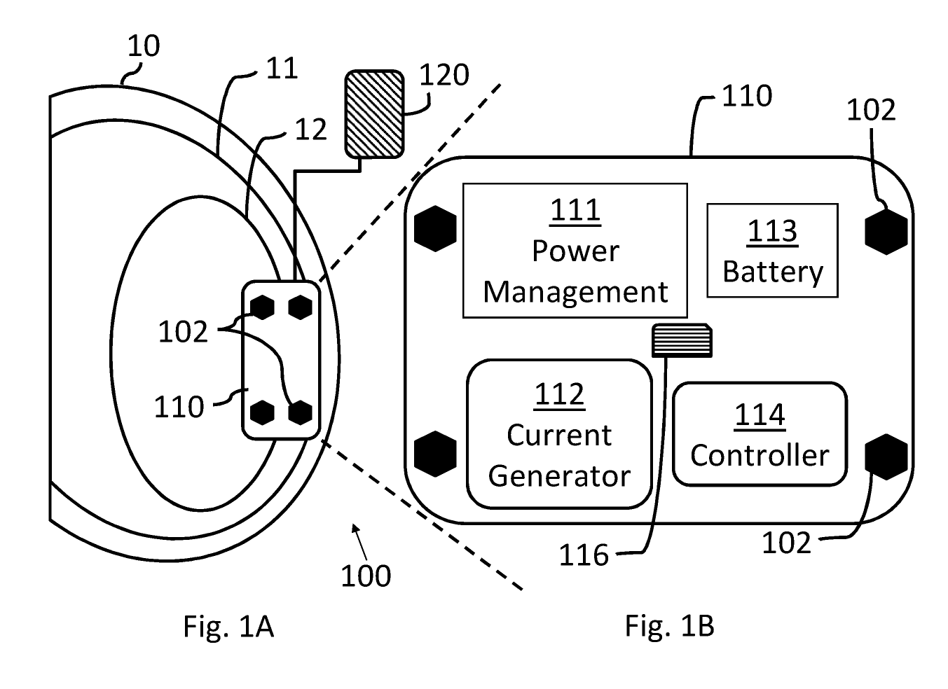

Reference is now made to FIGS. 1A and 1B, which show a contraction detection system 100 according to some embodiments. FIG. 1A schematically illustrates contraction detection system 100 for monitoring muscle (e.g. uterine) contraction of a patient 10, according to some embodiments of the invention. Contraction detection system 100 may be configured to allow monitoring of the abdomen of user 10 so as to detect contraction of the uterine wall.

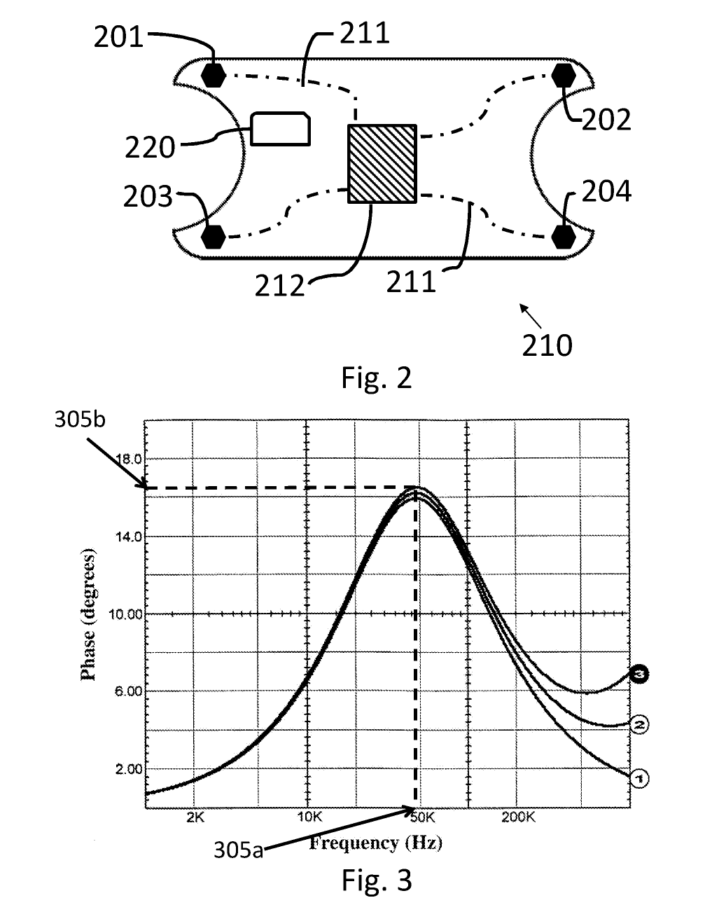

Contraction detection system 100 may comprise a monitoring unit 110 removably attachable to the body of user 10. In some embodiments, monitoring unit 110 may be a wearable element or a patch (e.g. as shown in FIG. 2) with an adhesive so as to attach monitoring unit 110 to the body. It should be appreciated that monitoring unit 110 may be attached or otherwise mounted on a user's abdomen in any other way known in the art.

According to some embodiments, monitoring unit 110 may include at least two electrodes 102 that may be configured to allow measurement of electric current passing through the abdomen of user 10 and thereby monitor uterine wall contractions. In some embodiments, electrodes 102 may be configured to be removably attachable and positioned apart from each other over an abdominal surface of a user 10. According to some embodiments, when placed over an abdominal surface of user 10, electrodes 102 may be positioned in a predetermined and fixed distance from each other. According to one embodiment electrodes 102 may be placed at least 5 centimeters (cm) apart from each other.

According to some embodiments, when monitoring unit 110 is placed over an abdominal surface of user 10, current generated by a current generator 112, may be conducted from one of electrodes 102 to another electrode 102 through different tissue layers. As the resistance of the muscle tissue (e.g. the uterine wall and abdomen muscles) is lower than the resistance of the skin and fat tissues, most of the current would pass through the muscle tissue of the abdomen of user 10. It should be appreciated by those skilled in the art that due to the resistance of the tissue, a voltage drop may be expected between electrodes 102. According to some embodiments, the voltage drop between electrodes 102 may be used to determine the bio-impedance of the muscle tissue. It should be noted that while the following description refers to bio-impedance, other electric parameters (such as phase) and signal behavior may also be utilized in order to detect contractions.

According to some embodiments, contraction detection system 100 may further comprise an input/output device 120 coupled to monitoring unit 110 and configured to display monitoring data from monitoring unit 110, for instance using a display unit such as a touchscreen or any other display. In some embodiments, input/output device 120 may further comprise a user interface unit. It should be appreciated that communication between monitoring unit 110 and input/output device 120 may be carried out through a wired connection, or alternatively through wireless communication, for instance using Infrared communication, Bluetooth, Wi-Fi communication, or any other wireless communication.

In some embodiments, input/output device 120 may further comprise an external processing unit (not shown) configured to allow processing of data gathered from monitoring unit 110.

According to some embodiments, input/output device 120 may be configured to present at least one of: uterine contraction occurrence, uterine contraction intensity, and uterine contraction duration. According to some embodiments, the intensity of a contraction may be determined according to the magnitude of change in the monitored parameter. For example, the larger the drop in bio-impedance or the change in phase during a contraction, the more intense the contraction may be. It should be appreciated that the duration of a contraction may be measured from the detection of a change in a monitored parameter indicative of a contraction until another change is detected that is indicative of retraction of muscle, such as, for example, return of the changed parameter to a level similar to the one measured prior to the detected contraction. For example, if the voltage drop between the electrodes 102 prior to a contraction was 6.5V and during the contraction the voltage drop changed to 5.5V, another change in the voltage drop between the electrodes 102 back to a value similar to 6.5V (e.g. between 6.2 and 6.7 volts) may indicate that the contraction has ended.

In some embodiments, contraction detection system 100 may also monitor the heart rate of a fetus 12 within uterus 11, using a dedicated sensor such as a Doppler sensor (not shown). Thus, for example, allowing the system to perform full fetal monitoring as is required for NST (Non Stress Test).

Reference is made to FIG. 1B which illustrates an enlarged section of monitoring unit 110. As may be seen in FIG. 1B, in addition to electrodes 102, monitoring unit 110 may comprise a current generator 112, a processor and/or controller 114, and a power storage unit (e.g. a battery) 113. According to some embodiments, current generator 112 may generate an alternating current (AC) in a predetermined frequency between the at least two electrodes 102. For example, the frequency of the generated alternating current may be in the range of 10-100 KHz. It should be appreciated that electrodes 102 are configured to allow detection and measurement of the current generated by current generator 112 passing through the body of user 10.

In some embodiments, in order to pass such a current (and also detect it and measure the output) through the abdominal surface of the user, electrodes 102 may be in contact (e.g. electrical contact) with the skin of user 10 and the generated current may pass from current generator 112 to electrodes 102 and from electrodes 102 pass the abdominal tissue of user 10. According to one embodiment an alternating current of 1 milliamper (mA) may be generated by current generator 112. The typical bio-impedance of blood may be about 100 Ohm/cm, muscle bio-impedance may be about 200 Ohm/cm. Fat may have bio-impedance of about 2000 Ohm/cm and skin may range from 1000-10000 Ohm/cm.

According to some embodiments, processor and/or controller 114 may be operatively coupled to the at least two electrodes 102 and configured to measure a voltage drop between the at least two electrodes 102 along time, wherein the voltage drop may correspond to interaction with tissues of the abdomen of user 10. According to some embodiments, a change in bio-impedance in the range of 4 to 18 percent may be indicative of a contraction. The resistance of tissue depends on the type of tissue as well as other properties of the tissue. For example, blood is typically about 100 Ohm/CM, muscle is about 200 Ohm/CM, fat is about 2000 Ohm/CM and skin may range anywhere between 1000-10000 Ohm/CM. Thus, for example, when placing electrodes 102, so that a current passing through the abdominal tissue between the electrodes pass through a total of 10 cm of tissue, (2 mm skin, 2 cm fat and 8 cm muscle) the resistance would be about 0.2*5000+2*2000+8*200=6,600 Ohm. If for example, the current passing between the electrodes and through the tissue is of 1 milliAmper (mA) the potential between the electrodes would be 6.6V. When the muscle tissue through which the current is passing contracts, the resistance per centimeter drops, for example to 150 Ohm per cm. Thus the total resistance drops by 400 Ohm (50×8 cm) and the potential between the electrodes would be only 6.2V. The change of 0.4V which is measured by monitoring unit 110 may be reported as a muscle contraction.

In some embodiments, processor and/or controller 114 may compare the phase (and/or other current related parameters) of the generated current and the current received by electrodes 102, in order to determine the occurrence of a muscle contraction For example, a change in phase from 12 degrees to 9 degrees may be indicative of a contraction.

Processor and/or controller 114 may further calculate a first bio-impedance value according to a first voltage drop and at least a second bio-impedance value according to at least a second voltage drop. In some embodiments, processor and/or controller 114 may determine occurrence of uterine contractions based on a change in bio-impedance between the bio-impedance values. In some embodiments, processor and/or controller 114 may continuously or repeatedly (e.g. every 10 milliseconds (ms), every 100 ms etc.) calculate impedance and phase difference from the measured bio-impedance values to detect a change (e.g. decrease) in the values indicative of a uterine contraction.

It should be appreciated that system 100 may be calibrated with predefined and/or measured base values for impedance and phase values, such that upon a difference in impedance and/or phase exceeding a predetermined threshold an indication of a uterine contraction may be provided. In some embodiments, uterine contraction intensity and duration may be measured and/or determined.

In some embodiments, measurements derived by controller 114 may be recorded and stored on a dedicated memory unit. Such a memory unit may be embedded into monitoring unit 110, or alternatively embedded into input/output device 120. In some embodiments, processor and/or controller 114 may alert user 10 or another user (such as a caretaker) of system 100, upon detection of a uterine contraction, for instance displaying an alert on a display of device 120.

According to some embodiments, monitoring unit 110 may further comprise a power management unit 111 that may be configured to control electrical power distribution between current generator 112 and power storage unit 113. In some embodiments, current generator 112 may comprise a digitally controlled oscillator (DCO) to control the amplitude and frequency of the generated signal. In some embodiments, processor and/or controller 114 may further comprise an analog to digital converter that may be configured to convert analog signals from the measured current to digital signals for controller 114, or vice versa.

According to some embodiments, monitoring unit 110 may further comprise a communication module that is configured to allow communication with output device 120. For instance, such communication module may facilitate wireless communication via Bluetooth low energy (BLE) unit or any other wireless communication unit known in the art.

According to some embodiments, monitoring unit 110 may further comprise at least one motion sensor 116 operatively coupled to the electrodes and configured to allow detection of movement of user 10. Motion sensor 116 may detect changes in position, inclination, acceleration, of monitoring unit 110 in order to identify non-uterine related muscle contraction caused by movement (e.g. change in position) of the monitored user 10. In some embodiments, the at least one motion sensor 116 may be selected from a group consisting of: accelerometer, gyroscope and microelectromechanical sensors.

Reference is now made to FIG. 2, which schematically illustrates monitoring unit as a disposable patch 210, according to some embodiments of the invention. Patch 210 may be configured to removably attach to the abdomen of user 10 with at least two electrodes 201-204 measuring current so as to detect uterine contractions. It should be appreciated that disposable patch 210 may be easily applied by any user onto any portion of the abdominal surface of the user, and even by the user herself, since all that is required is the application of a patch onto the skin. According to some embodiments, patch 210 is configured to maintain a predefined and fixed distance between at least two electrodes 201, 202, 203 and 204.

Patch 210 may further comprise a central controlling unit 212 that may be operably coupled to electrodes 201-204, with a wired connection 211. Central controlling unit 212 may control the generation and detection of the electrical signal, and other functions of the monitoring unit, for instance central controlling unit 212 may control communication with output device 120 (e.g. via wireless communication).

According to some embodiments, monitoring unit of disposable patch 210 may further comprise at least one motion sensor 220 operatively coupled to the electrodes and configured to allow detection of movement as described above with respect to FIGS. 1A and 1B. It should be appreciated that utilizing motion detection by at least one motion sensor 220, it may be possible to eliminate false contraction signals that are caused by movement.

Abdominal muscle tissue has directional voluntary muscles that contract in order to allow movement of the body. For instance, the Rectus abdominis muscles contract substantially along a longitudinal axis of the user (from head to toe). Since uterine wall muscles are smooth and non-directional, the contraction of the uterine wall may be identified in more than one contraction directions. Thus, a uterine contraction may be differentiated from a voluntary abdomen muscle contraction by measuring of contraction in more than one direction along the abdomen of user 10.

According to some embodiments, patch 210 may comprise three or more electrodes in order to allow determining by a controller, such as controller 114 (in FIG. 1B) or a processor in input/output device 120 (in FIG. 1A), bio-impedance changes along at least two directions, and determine that an identified contraction is a uterine contraction when contractions are measured in more than one direction, or determine that an identified contraction is not an uterine contraction when a contraction is identified in a single direction.

Furthermore, since abdominal muscles contract as a single unit and uterine muscles contract as a longitudinal wave (similar to peristaltic contraction) beginning around the upper (e.g. closer to the chest) ventral region of the torso and moving towards the direction of the pelvis, along two different portions of the abdominal surface, the first portion closer to the upper ventral region of the torso and the second closer to the lower ventral region of the torso (closer to the pelvis), a uterine contraction may be identified first at the upper portion and after a substantially fixed time interval at the lower position.

According to some embodiments, a first pair of electrodes (e.g. electrodes 201 and 202) may be configured to detect a contraction in a direction orthogonal to the longitudinal axis of the user (i.e. from head to toe) at the first portion of the abdominal surface of the user. The first portion may be located, for example, along the midriff of user 10. According to some embodiments, a second pair of electrodes (e.g. electrodes 203 and 204) may be configured to detect a contraction in substantially the same direction (orthogonal to the longitudinal axis of the user) at the second portion of the abdominal surface of the user. The second position may be located, for example, along an imaginary line parallel to the midriff of user 10, and closer to the pelvis then the first position. According to some embodiments, a processor, such as processor or controller 114, may determine that a contraction is a uterine contraction if measurements of bio-impedance change indicative of muscle contraction are received from the first pair of electrodes (201 and 202) while, for example, no corresponding contraction is measured by the second pair of electrodes, and after a predefined time interval a corresponding contraction is measured by the second pair of electrodes. In the scope of this application the term ‘corresponding contraction’ may refer to a contraction with similar direction and magnitude (e.g. intensity) as another contraction to which it corresponds. It should be appreciated that the time interval may be affected, inter alia, by one or more of: the distance along the longitudinal axis of the user 10 between the first pair of electrodes and the second pair of electrodes, the posture and position of user 10, the intensity of the contraction and the like.

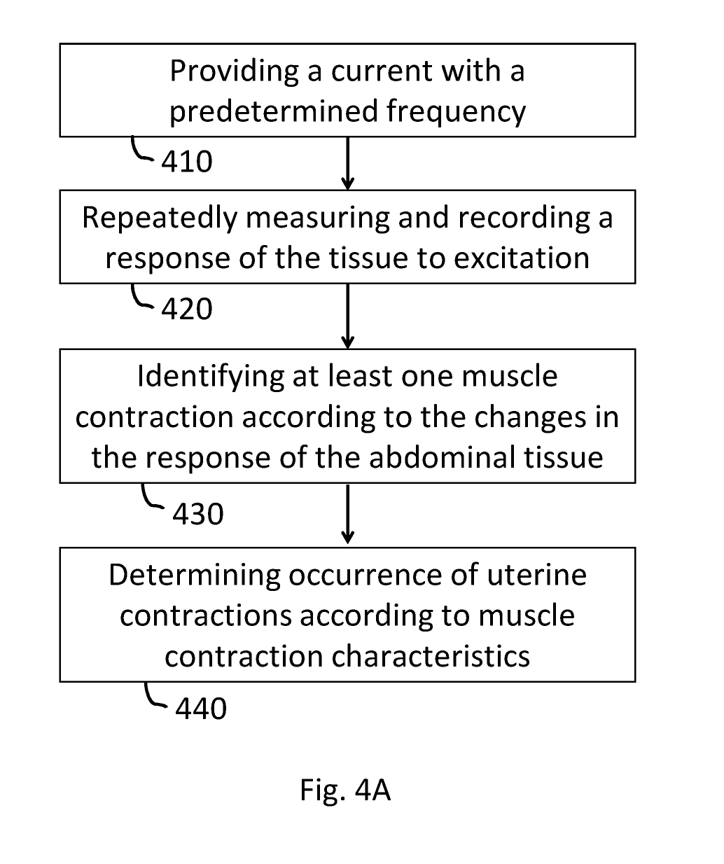

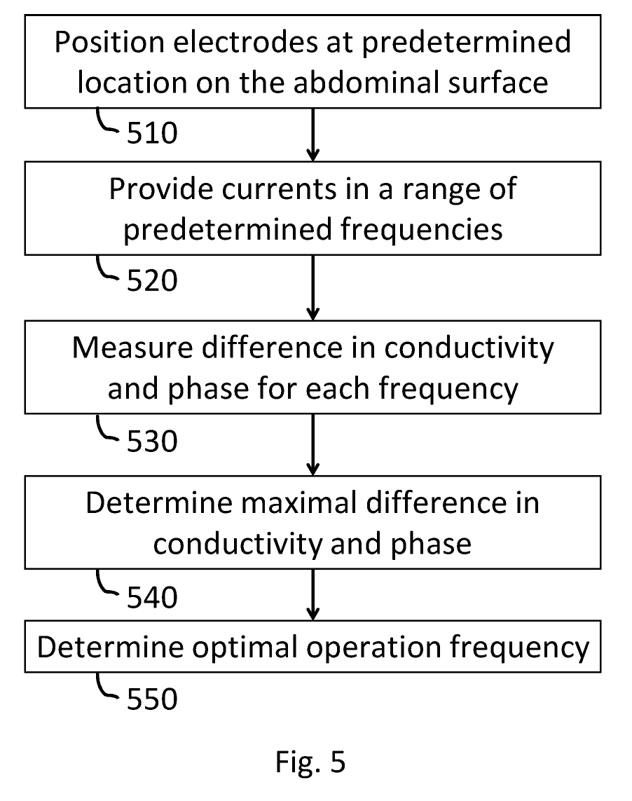

Reference is now made to FIG. 3, which is a graph showing examples of phase changes as function of frequency and location of electrodes over the abdominal surface, according to some embodiments of the present invention. It should be noted that when current in the optimal operation frequency 305a passes the abdominal tissue (due to the generated electric current), the difference in conductivity and phase, per biological mass unit, is maximal 305b. The frequency for such optimal performance may depend on the individual physiology, temperature, hydration levels and other parameters of user 10, as well as the position of the electrodes. For example, the optimal frequency may be in the range of 10-100 KHz. Therefore, measuring the difference in phase may allow determination of the optimal operation frequency for each user. As illustrated in FIG. 3, measurements have been performed at three different locations over a user's abdomen and for each location an alternating current in different frequencies have been applied. FIG. 3 shows the changes in phase as a function of the changes in frequency for each of three locations 1, 2 and 3. In some embodiments, an initial calibration process may be carried out for each user with measurement of the difference in phase as described with reference to FIG. 5 herein, in order to determine the optimal frequency that may provide increased accuracy of detecting uterine contractions. According to some embodiments such calibration may be conducted before each use, periodically throughout the testing session (e.g. the contraction detection session).

As seen in the graph of FIG. 3, the maximum change in phase 305b is reached, for a specific tested user, at about 48 kHz. Accordingly, in this specific instance, it may be realized that the optimal operation frequency 305a for that user is 48 kHz. It should be appreciated that while different locations 1, 2 and 3, resulted in a different change in phase, the change in location did not affect the optimal operation frequency 305a. According to some embodiments, the preferred location to place the electrodes may be location 3 as at this location, the measured change if phase for all frequencies is maximal 305b, for the texted user. It should be appreciated that for other users and/or in other conditions and locations, other optimal operation frequencies and other maximal phase changes may be measured.

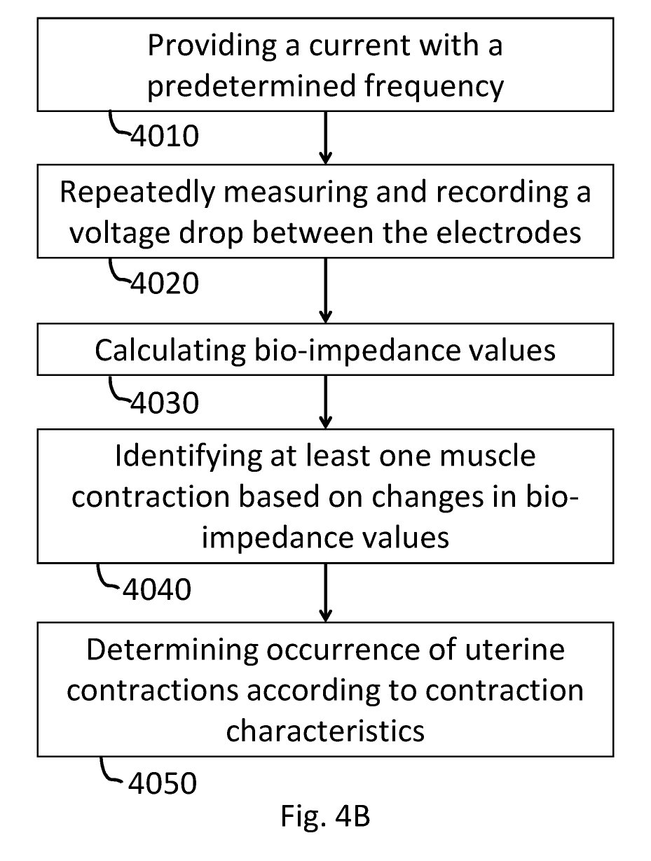

Reference is now made to FIG. 4A, which is a flowchart for a method of detecting uterine contractions, according to some embodiments of the invention. According to some embodiments, initially, an electrical current (AC) with a predetermined frequency may be provided 410, for instance generated with a current generator and provided through a first path between at least a first pair of electrodes positioned apart from each other over a first abdominal surface of the user.

According to some embodiments, a processor of monitoring device 110, may repeatedly measure and record a response 420 of the abdominal tissue to excitation by the alternating current passing between the at least first pair of electrodes, and may identify 430 at least one muscle contraction according to the changes in the response of the abdominal tissue. According to some embodiments, the response may be measured in at least one of bio-impedance values, reactance values and phase.

According to some embodiments, the processor (114 in FIG. 1) may determine 440 occurrence of uterine contractions based on at least one characteristic of the at least one muscle contraction. The characteristics of a muscle contraction may refer to direction or directions of contraction, intensity of contraction, duration of contraction, repetitiveness of contraction and the like.

According to some embodiments, such a method of detection of uterine contractions may further comprise determination of at least a first and second muscle contraction values, the contraction values indicative of a magnitude (e.g. the intensity, duration etc.) of contraction of the muscle in a first and second direction, respectively, and determination of occurrence of uterine contraction when the first value is bigger than a first contraction threshold value, and the at least second contraction values is bigger than a second contraction threshold value.

According to some embodiments, such a method of detection of uterine contractions may further comprise comparison of at least the first muscle contraction value and the second contraction value, and determining occurrence of uterine contraction when the difference between the first contraction value and the second contraction value is smaller than a predefined value.

According to some embodiments, occurrence of a muscle contraction may be determined when the change in response of the abdominal tissue indicates a decrease in bio-impedance between the electrodes of the at least first pair of electrodes. According to some embodiments, an indication on an output device may be presented, to indicate at least one of: an occurrence of a uterine contraction, an intensity of the uterine contraction, and duration of the uterine contraction. According to some embodiments, time between contractions may also be calculated, stored in a memory and may be presented on an output device, such as a screen of a mobile computing device (e.g. a smartphone, a tablet computer and the like).

According to some embodiments, a method of detection of uterine contractions may further comprise monitoring movement of the user, with at least one motion sensor, and calculating a movement intensity value, wherein the calculation of the movement intensity value may be based on at least one of: acceleration of the at least one motion sensor and change in inclination of the at least one sensor. In some embodiments, the determination of occurrence of uterine contraction may further require that the calculated movement intensity value is within a predefined range.

According to some embodiments, such a method of detection of uterine contractions may further comprise providing the alternating current with the determined frequency, over a second path between at least a second pair of electrodes positioned apart from each other over a second abdominal surface of the user, wherein the second abdominal surface may be closer to the pelvis of the user than the first abdominal surface, and wherein the at least first pair of electrodes and the at least second pair of electrodes may comprise at least three electrodes. In some embodiments, the change in bio-impedance over the second path may be calculated along time.

In some embodiments, occurrence of uterine contractions may be determined where the change in response of the tissue over the first abdominal surface may be indicative of uterine contraction, and the change in response over the second abdominal surface may be indicative of uterine contraction. In some embodiments, the change in response over the first abdominal surface may occur at a predefined time interval prior to the change in bio-impedance over the second abdominal surface.

Reference is made to FIG. 4B which is an example of method of detecting uterine contractions based on changes in bio-impedance, according to some embodiments of the present invention. Initially, an electrical current (AC) with a predetermined frequency may be provided 4010, for instance generated with a current generator and provided through a first path between at least a first pair of electrodes positioned apart from each other over a first abdominal surface of the user.

Next, a voltage drop may be repeatedly measured and recorded 4020 between the at least two electrodes of the at least first pair of electrodes, for instance measured with a processor or controller along time.

According to some embodiments, a first bio-impedance value may be calculated 4030 according to a first voltage drop and at least a second bio-impedance value may be calculated (e.g. by processor or controller 114 in FIG. 1B) according to at least a second voltage drop.

According to some embodiments, at least one muscle contraction may be identified 4040 (e.g. by processor or controller 114) according to the changes in bio-impedance between the first bio-impedance value and the at least second bio-impedance value.

After a muscle contraction has been identified, the occurrence of uterine contractions may be determined 4050 (e.g. by controller 114) based on at least one characteristic of the at least one muscle contraction. According to some embodiments, characteristics of the muscle contraction may include one or more of the direction and/or directions of contraction of the muscle, the intensity of contraction, the duration of contraction, the repetitiveness of contraction and/or any combination thereof.

It should be appreciated that while the above example refers to changes in bio-impedance to determine muscle contraction, other parameters of the tissue or the current may be used, such as, for example changes in the phase of the alternating current and/or reactance of the tissue.

It should be noted that current studies show that as muscle tissue has fibers with blood vessels and various fluids, contraction of the muscle may cause “squeezing” of these liquids and thereby increase resistance since liquids have a higher conductivity than muscle tissue. In contrast to such studies, it may be shown that during muscle contraction, the resistance between two electrodes attached to the skin actually decreases. It should be appreciated that during muscle contraction, the resistance of each fiber may increase, while the amount of fibers between two points also increases due to thickening of the muscle. Therefore, similarly to resistors coupled in parallel where the resistance is divided between the resistors, the resistance during muscle contraction may also decrease. It should be appreciated that such observation of muscle contraction may further assist in uterine contraction detection, where false contractions and other noise factors may be eliminated.

It should be appreciated that such a way of detecting uterine contractions does not require professional training and may be performed by anyone (including the user), at any location on the abdominal surface, for instance used for domestic monitoring at all stages of pregnancy (e.g. detecting uterine contractions from 23rd week). Furthermore, such a system may allow motion during measurements since movement of the user may not disrupt the measurement due to the motion sensor eliminating signals caused by movement or voluntary muscle contraction. Moreover, such a system may detect uterine contractions regardless of percentage of fat in the body of the user, since the current passes mainly through the uterine wall due to the lower resistance of muscle tissue than fat and skin tissues.

Reference is made to FIG. 5, which shows a flowchart of a method of frequency calibration, according to some embodiments of the present invention.

According to some embodiments, such a method may include providing at least a first alternating current with a first frequency and a second alternating current with a second frequency, between two electrodes of the at least first pair of electrodes, and for each of the at least first and second frequencies, calculating phase difference between the alternating current provided at a first electrode of the at least first pair of electrodes and the alternating current received at a second electrode of the at least first pair of electrodes. Then, a frequency may be selected from the at least first and second frequencies, for which the calculated phase difference is maximal, as the determined frequency of operation. In some embodiments, the determined frequency may be re-determined for each measurement session.

As seen in FIG. 5, initially, at least two electrodes (e.g. electrodes 102) may be positioned 510 at predetermined locations on the abdominal surface of a user. It should be noted that since the distance between the electrodes may affect the measurement, a predetermined distance may be initially selected and set. According to some embodiments, the electrodes may be embedded in a patch in order to maintain the fixed distance and relative location of the electrodes with respect to each other and/or other components of the monitoring unit.

After the electrodes are placed, a current in a range of predetermined frequencies may be provided 520 (e.g. with current generator 112) to at least one of the electrodes and conducted through tissue of the user to the at least second electrode. For each of these frequencies, the difference between the provided signal parameters (e.g. the phase of the current provided) and the received signal may be measured 530.

According to some embodiments, a processor of device 100 may identify the frequency in which the maximal difference in signal parameters is measured. Finally, an optimal operation frequency may be determined 550, corresponding to the maximal determined difference in signal parameters (e.g. conductivity and phase). It should be appreciated that the calculation of maximal difference in conductivity and phase may also correspond to the distance between the electrodes and other physiological features of the user, such that each user may receive an individual optimal operation frequency and may have different individual optimal operation frequency in different operation sessions.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of detecting contractions in a user, the method comprising:
providing alternating current with a determined frequency, through a first path in an abdominal tissue, between at least a first pair of electrodes positioned apart from each other over a first abdominal surface of the user;
repeatedly measuring a response of the abdominal tissue to excitation by the alternating current passing between the at least first pair of electrodes;
identifying at least one muscle contraction according to the changes in the response; and
determining occurrence of uterine contractions based on at least one characteristic of the at least one muscle contraction.

2. The method according to claim 1, wherein the response is measured in at least one of bio-impedance value, reactance value and current phase.

3. The method according to claim 1, further comprising repeatedly measuring and recording a voltage drop between at least two electrodes of the at least first pair of electrodes, through time.

4. The method according to claim 1, wherein the first path is perpendicular to a longitudinal axis of a user wherein the longitudinal axis is directed from the top of the head of the user towards the pelvis of the user.

5. The method of claim 1, further comprising:
determining at least a first muscle contraction value, the first contraction value indicative of a magnitude of contraction of the muscle in a first direction;
determining at least a second muscle contraction value, the second contraction value indicative of the magnitude of contraction of the muscle in a second direction; and
determining occurrence of uterine contraction when the first value is bigger than a first contraction threshold value, and the at least second contraction value is bigger than a second contraction threshold value.

6. The method of claim 1, further comprising:
determining at least a first muscle contraction value, the first contraction value indicative of a magnitude of contraction of the muscle in a first direction;
determining at least a second muscle contraction value, the second contraction value indicative of the magnitude of contraction of the muscle in a second direction;
comparing at least the first muscle contraction value and the second muscle contraction value; and
determining occurrence of uterine contraction when the difference between the first contraction value and the second contraction value is smaller than a predefined value.

7. The method of claim 1, wherein determining the frequency comprises:
providing at least a first alternating current with a first frequency, between the two electrodes of the at least first pair of electrodes;
providing at least a second alternating current with a second frequency, between two electrodes of the at least first pair of electrodes;
for each of the at least first and second frequencies, calculating phase difference between the alternating current provided at a first electrode of the at least first pair of electrodes and the alternating current received at a second electrode of the at least first pair of electrodes; and
selecting a frequency from the at least first and second frequency, for which the calculated phase difference is maximal, as the determined frequency.

8. The method of claim 1, wherein the determined frequency is in the range of 10 kHz-100 kHz.

9. The method of claim 1, wherein the determining of the frequency is repeated every predefined time interval.

10. The method of claim 1, wherein the response is measured in bio-impedance values, and wherein occurrence of a muscle contraction is determined when the change in bio-impedance between a first bio-impedance value and the at least one second bio-impedance value indicates a decrease in bio-impedance between the electrodes of the at least first pair of electrodes.

11. The method of claim 1, further comprising determining uterine contraction intensity and duration.

12. The method of claim 11, further comprising presenting an indication on an output device, to indicate at least one of: an occurrence of a uterine contraction, an intensity of the uterine contraction, and a duration of the uterine contraction.

13. The method of claim 1, further comprising:
monitoring movement of the user, with at least one motion sensor;
calculating a movement intensity value, wherein the calculation of the movement intensity value is based on at least one of: acceleration of the at least one motion sensor and change in inclination of the at least one motion sensor; and
wherein the determination of occurrence of uterine contraction further requires that the calculated movement intensity value is within a predefined range.

14. The method of claim 1, wherein the determination of occurrence of uterine contraction further requires:
providing the alternating current with the determined frequency, over a second path between at least a second pair of electrodes positioned apart from each other over a second abdominal surface of the user, wherein the second abdominal surface is closer to the pelvis of the user than the first abdominal surface;
calculating a change in tissue response over the second path along time; and
determining occurrence of uterine contractions when:
the change in response over the first path is indicative of uterine contraction;
the change in response over the second path is indicative of uterine contraction; and
the change in response over the first path occurs at a predefined time interval prior to the change in response over the second path.

15. A contraction detection system, comprising:
an alternating current generator configured to provide an alternating current in a determined frequency, through a first path in an abdominal tissue, between at least two electrodes configured to be positioned apart from each other over an abdominal surface of a user;
and
a processor, operatively coupled to the at least two electrodes, the processor configured to:
repeatedly measure and record on a memory, a response of the abdominal tissue to excitation by the alternating current passing between the at least two electrodes;
identify at least one muscle contraction according to the changes in the response; and
determine occurrence of uterine contractions based on at least one characteristic of the at least one muscle contraction.

16. The system of claim 15, further comprising at least one motion sensor, operatively coupled to the at least two electrodes and configured to allow detection of movement of the user.

17. The system of claim 16, wherein the at least one motion sensor is selected from a group consisting of accelerometer, gyroscope and microelectromechanical sensor.

18. The system of claim 15, further comprising an output device, in active communication with the processor, the output device configured to present at least one of: uterine contraction occurrence, uterine contraction intensity, and uterine contraction duration.

19. The system according to claim 18, wherein the output device is a mobile device, connected to the processor via a wireless communication channel.

20. The system of claim 15, wherein the at least two electrodes are removably attachable to an abdominal surface of a user.

21. (canceled)

22. (canceled)
